(19) United States
(12) Patent Application Publication
(10) Pub. No.: US 2011/0224559 A1
(43) Pub. Date: Sep. 15, 2011

(54) HIGH-ACCURACY HEMADYNAMOMETER AND METHOD OF USING THE SAME

(76) Inventor: Yang Cheng CHEN, Zhubei City (TW)

(21) Appl. No.: 12/853,828

(22) Filed: Aug. 10, 2010

(30) Foreign Application Priority Data
Mar. 10, 2010 (TW) 099106888

(51) Int. Cl. A61B 5/0225

(52) U.S. Cl. 600/493

(57) ABSTRACT

The invention provides a high-accuracy hemadynamometer and method of using the same. A main air tank imposes pressure upon a wrist or an arm of a user. There is a valve between an auxiliary air tank and the main air tank to release air in the auxiliary air tank to the main air tank. A pressurized device increases pressure of the main air tank and the auxiliary air tank to impose pressure upon the wrist or the arm of the user. A pressure release device is set on the main air tank. A pressure detector detects pressure of the main air tank and outputs a pressure value. A heartbeat detector detects the oppression of the blood of the artery of the user and outputs a pulsation signal. A controller controls the pressure release device to release air in the main air tank and activates the valve based on the pulsation signal for releasing air in the auxiliary air tank to the main air tank.

### BACKGROUND OF THE INVENTION

1. Field of the Invention

The invention relates generally to a hemadynamometer and, more particularly, to a hemadynamometer capable of releasing air in an auxiliary air tank into a main air tank for accurately measuring the systolic blood pressure and the diastolic blood pressure of a user.

2. Description of Related Art

According to statistic data, the aging of population is getting more and more serious. As the average age of family members increases gradually, cardiovascular diseases will become one of the important issues of medical prevention in the future. In order to prevent cardiovascular diseases as early as possible, a hemadynamometer is an indispensable basic measuring device in every family.

Today, current used hemadynamometers are divided into manual-pump type hemadynamometers, sound type hemadynamometers and oscillation blood pressure type hemadynamometers. The manual-pump type hemadynamometer comprises an arm band, a manual pump, an air release valve, a mercury manometer and a stethoscope. Doctors usually have manual-pump type hemadynamometers for diagnosing. When the manual-pump type hemadynamometer is used to measure blood pressure, the arm band containing an air tank is tied to the upper arm of a person for testing blood pressure. The doctor presses manual-pump to pump air into the air tank, so as to inflate it and increase pressure in it. As the arm band presses the artery of the upper arm of the person for blocking the blood from flowing through the upper arm temporarily, the doctor reads the pressure value according to the mercury manometer. Then, the doctor opens the air release valve connected to the arm band to slowly decrease the pressure in the arm band. When the pressure is slightly lower than the systolic blood pressure of the artery, the blood in the artery generates Korothov sound from the eddy current of the arm band pressed position. Then, the doctor can distinguish this sound from the stethoscope arranged on the artery of the upper arm. The measured pressure is called systolic blood pressure. As the pressure in the arm band decreases, the doctor can hear the Korothov sound continuously. After Korothov sound disappears, the measured pressure is known as diastolic blood pressure.

The sound type hemadynamometer measures the pulse amplitude of a user and transforms it into pulse amplitude voltage signal. This pulse amplitude voltage signal is compared with a reference voltage, e.g. 0.5 V, to generate a beep sound when the pulse amplitude voltage signal is higher than 0.5 V. The pressure value corresponding to the pulse of the first beep sound is known as systolic blood pressure.. After continuous beep sound disappears, the pressure value corresponding to the pulse of the last beep sound is the diastolic blood pressure.

The oscillation blood pressure type hemadynamometer detects the maximum amplitude Amax in the oscillation blood pressure first, and then searches forward for the position of the 0.5 Amax (based on a statistic method). The pressure value corresponding to this 0.5 Amax is the systolic blood pressure. Then, the oscillation blood pressure type hemadynamometer searches backward for the position of the 0.8 Amax (also based on a statistic method), and the pressure value corresponding to this 0.8 Amax is the diastolic blood pressure.

No matter what kind of the hemadynamometer is used, a wrist/arm band containing an air tank is tied to the upper arm or the wrist of a user, and a manual pump or an air compressed pump is used to pump air into the air tank in the wrist/arm band. When the pressure imposed is higher than a pre-set value (i.e. 220 mmHg) of a standard systolic pressure (i.e. 140 mmHg) of a normal person, the wrist/arm band presses the artery of the wrist or the arm of the user for blocking the blood from flowing through the wrist or the arm temporarily.

Then, a pressure release device of the air tank is used to decrease the pressure in the air tank slowly. When the pressure is slightly lower than the systolic blood pressure of the artery, the blood in the artery generates Korothov sound from the eddy current of the wrist/arm band pressed position for detecting systolic blood pressure value. FIG. 1 illustrates a pressure diagram of air being released from an air tank in the prior art.

As shown in FIG. 1, the pressure decreases with a slope when air in the air tank is released. If the slope is set lower than average, it corresponds to less air in the air tank being released. As the speed of air release is lower, the accuracy of the systolic blood pressure and the diastolic blood pressure is better. However, it takes much time to get it. If the slope is set to be larger than average, it corresponds to more air in the air tank being released. Although the measuring time can be shortened, the speed of air release is higher while the accuracy of the systolic blood pressure and the diastolic blood pressure is getting worse.

Another method is to use the process illustrated in FIG. 1 to measure an approximate systolic blood pressure or an approximate diastolic blood pressure and then measure again for the second time. In the second measurement, the release slope of the air tank is set to be smaller at the point close to the approximate systolic blood pressure or the approximate diastolic blood pressure. The air in the air tank is released slowly for measuring systolic blood pressure and diastolic blood pressure more accurately. However, this method takes much time. Therefore, it is desired for the above hemadynamometers and their measuring method to be improved.

### SUMMARY OF THE INVENTION

The object of the present invention is to provide a high-accuracy hemadynamometer, wherein its main air tank can be used to decrease the pressure inside the hemadynamometer with fast speed so as to save measuring time. The pressure close to the diastolic blood pressure and systolic pressure of the user in the main air tank can be increased with slower speed to measure the blood pressure of the user accurately. At the same time, an auxiliary air tank can be used to avoid additional power loss resulting from frequent starts of a pressurized device without generating noise. The present invention is especially suitable for battery-powered handheld hemadynamometer.

According to one aspect of the present invention, there is provided a high-accuracy hemadynamometer for measuring the blood pressure of a user, which comprises: a main air tank for imposing pressure upon a wrist or an arm of the user; an auxiliary air tank connected to the main air tank, wherein a valve is set between the auxiliary air tank and the main air tank to release air in the auxiliary air tank into the main air tank; a pressurized device connected to the main air tank and the auxiliary air tank for pumping air into the main air tank and the auxiliary air tank to impose pressure upon the wrist or the arm of the user; a pressure release device arranged on the main air tank; a pressure detector connected to the main air tank for detecting the pressure of the main air tank and outputting a pressure value; a heartbeat detector for detecting heartbeats of the user and generating a pulsation signal corresponding to the heartbeats; and a controller connected to the pressure detector, the heartbeat detector, the pressure release device and the valve for controlling the pressure release device to release the pressure in the main air tank, and activating the valve based on the pulsation signal to release air in the auxiliary air tank into the main air tank.

According to another aspect of the present invention, there is provided a high-accuracy blood pressure measuring method for measuring the blood pressure of a user, which comprises the steps of: (A) using a pressurized device to pump air into a main air tank and a auxiliary air tank up to a predetermined pressure value; (B) using a pressure release device of the main air tank to decrease the pressure in the main air tank with a first fast slope (S_{fast1}); (C) when a pulsation signal outputted by a heartbeat detector is Korothov sound, setting a pressure value outputted by a pressure detector as an approximate systolic pressure (Pₐ₋ₕₚ); (D) opening a valve to release air in the auxiliary air tank into the main air tank for increasing the pressure in the main air tank with a first slow slope (S_{slow1}), and when the Korothov sound disappears, setting the pressure value outputted by the pressure detector as an accurate systolic pressure (Pₕₚ), and closing the valve; (E) using the pressure release device to decrease the pressure in the main air tank with a second fast slope (S_{fast2}); (F) when the Korothov sound disappears again, setting the pressure value outputted by the pressure detector as an approximate diastolic blood pressure; (G) opening the valve to release air in the auxiliary air tank into the main air tank again for increasing the pressure in the main air tank with a second slow slope S_{slow2}) and when the Korothov sound appears, setting the pressure value outputted by the pressure detector as an accurate diastolic blood pressure, and closing the valve; and (H) releasing the pressure in the main air tank and the auxiliary air tank.

According to a further aspect of the present invention, there is provided a high-accuracy blood pressure measuring method for measuring the blood pressure of a user, which comprises the steps of: (A) using a pressurized device to pump air into a main air tank and a auxiliary air tank up to a predetermined pressure value; (B) using a pressure release device of the main air tank to decrease the pressure in the main air tank with a first fast slope (S_{fast1}); (C) when a pulsation signal outputted by a heartbeat detector is Korothov sound, setting a pressure value outputted by a pressure detector as an approximate systolic pressure (Pₐ₋ₕₚ); (D) opening a valve completely to release air in the auxiliary air tank into the main air tank quickly for increasing the pressure in the main air tank up to a first predetermined pressure value (P_{H}), and using a pressure release device to decrease the pressure in the main air tank with a first slow slope (S_{slow1}), and when the Korothov sound appears, setting the pressure value outputted by the pressure detector as an accurate systolic pressure (Pₕₚ), and closing the valve; (E) using the pressure release device to decrease the pressure in the main air tank with a second fast slope (S_{fast2}); (F) when the Korothov sound disappears again, setting the pressure value outputted by the pressure detector as an approximate diastolic blood pressure; (G) opening the valve completely to release air in the auxiliary air tank into the main air tank quickly for increasing the pressure in the main air tank up to a second predetermined pressure value (P_{H}), and using the pressure release device to decrease the pressure in the main air tank with a second slow slope (S_{slow2}), and when the Korothov sound disappears again, setting the pressure value outputted by the pressure detector as an accurate diastolic blood pressure (Pₕₚ), and closing the valve; and (H) releasing the pressure in the main air tank and the auxiliary air tank.

### BRIEF DESCRIPTION OF THE DRAWINGS

FIG. 1 illustrates a pressure diagram of air being released from an air tank in the prior art;

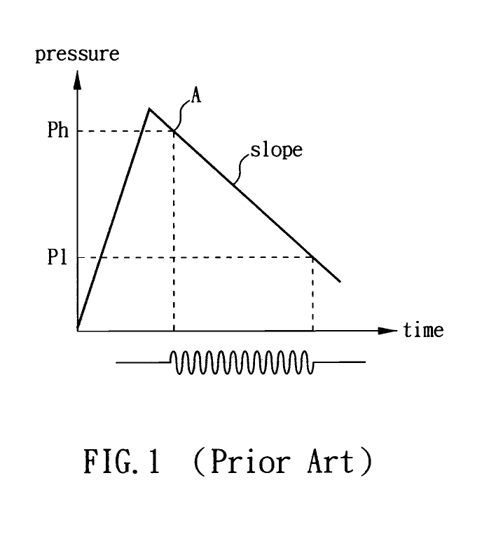

FIG. 2 shows a block diagram of a high-accuracy hemadynamometer in accordance with one embodiment of the present invention;

FIG. 3 shows a pressure diagram of air being released from the main air tank in accordance with one embodiment of the present invention;

FIG. 4 shows a flow chart of high-accuracy blood pressure measuring method in accordance with one embodiment of the present invention;

FIG. 5 shows a pressure diagram of air being released from the main air tank in accordance with another embodiment of the present invention; and

FIG. 6 shows a flow chart of high-accuracy blood pressure measuring method in accordance with another embodiment of the present invention.

### DETAILED DESCRIPTION OF THE PREFERRED EMBODIMENT

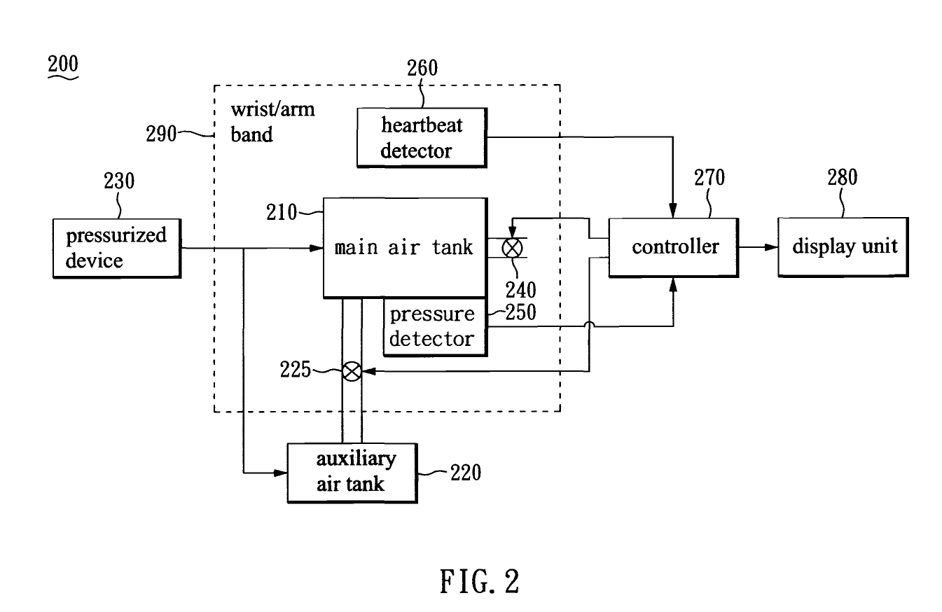

With reference to FIG. 2, there is shown a block diagram of a high-accuracy hemadynamometer 200 for measuring the blood pressure of a user in accordance with the present invention. The high-accuracy hemadynamometer 200 includes a main air tank 210, an auxiliary air tank 220, a pressurized device 230, a pressure release device 240, a pressure detector 250, a heartbeat detector 260, a controller 270 and a display unit 280.

The main air tank 210 is used for imposing pressure upon the wrist or the arm of a user. The auxiliary air tank 220 is connected to the main air tank 210, wherein a valve 225 is arranged between the auxiliary air tank 220 and the main air tank 210 for releasing air in the auxiliary air tank 220 into the main air tank 210.

The main air tank 210 can be arranged in a wrist/arm band 290, which is tied around the wrist/arm of the user.

The pressurized device 230 is connected to the main air tank 210 and the auxiliary air tank 220 for pumping air into the main air tank 210 and the auxiliary air tank 220 to impose pressure upon the wrist or the arm of the user. Preferably, the pressure release device 230 is a motor or an air pump. When the pressurized device 230 pumps air into the main air tank 210 and the auxiliary air tank 220 for imposing pressure, the valve 225 is open, and thus the pressure of the auxiliary air tank 220 is equal to the pressure of the main air tank 210.

The pressure release device 240 is arranged on the main air tank 210 for decreasing the pressure of the main air tank 210 of the pressure release process, so as to decrease the pressure of the wrist or the arm of the user imposed by the wrist/arm band 290. Preferably, the pressure release device 240 is an air releasing valve.

The pressure detector 250 is connected to the main air tank 210 for detecting the pressure of the main air tank 210 and outputting a pressure value.

The heartbeat detector 260 is used for detecting heartbeats of the user and generating a pulsation signal corresponding to the heartbeats. The heartbeat detector 260 can be arranged in the wrist/arm band 290 to generate a pulsation signal.

The controller 270 is connected to the pressure detector 250, the heartbeat detector 260, the pressure release device 240 and the valve 225 for controlling the pressure release device 240 to release the pressure in the main air tank 210, and activating the valve 225 based on the pulsation signal so as to release air in the auxiliary air tank 220 into the main air tank 210.

The display unit 280 is connected to the controller 270 to display the pressure value outputted by the pressure detector 250. The display unit 280 can be a liquid crystal display (LCD) or an organic light emitting diode (OLED). The auxiliary air tank 220 also can be arranged in the wrist/arm band 290. Due to that the auxiliary air tank 220 is made from a rigid container, the pressure of the auxiliary air tank 220 does not affect the pressure of the wrist/arm band 290 directly. Alternatively, the auxiliary air tank 220 may be not directly connected to the pressure release device 230. When the auxiliary air tank 220 pumps air into the main air tank 210, the valve 225 is open completely. Thus, the auxiliary air tank 220 can impose pressure upon the auxiliary air tank 220 via the main air tank 210 and the valve 225.

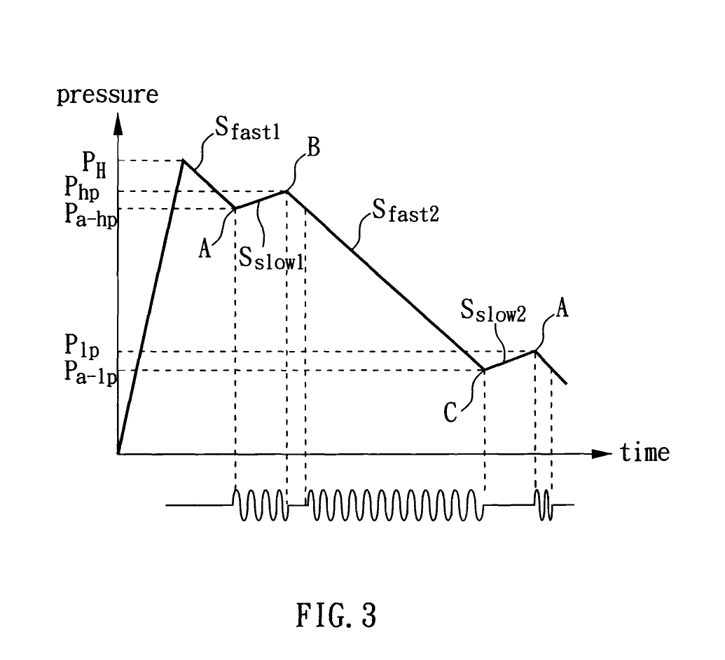

FIG. 3 shows a pressure diagram of air releasing in the main air tank 210 in accordance with the present invention. When measuring blood pressure, the wrist/arm band 290 is tied around the wrist or the arm of the user. The pressure release device 230 imposes pressure upon the main air tank 210 and the auxiliary air tank 220. When the pressure imposed by the pressure release device 230 is higher than a predetermined value P_{H} (i.e. 220 mmHg) relative to a standard systolic pressure (i.e. 140 mmHg) of a normal person, the wrist/arm band 290 presses the artery of the wrist or the arm of the user to block the blood flowing through the wrist or the arm temporarily.

Then, the controller 270 activates the pressure release device 240 to decrease the pressure in the main air tank 210 with a first fast slope S_{fast1}; that is, using the pressure release device 240 to decrease the pressure of the main air tank 210 in the wrist/arm band 290 with a first fast slope S_{fast1}. In the pressure release process, when the pressure of the main air tank 210 in the wrist/arm band 290 is higher than the blood pressure of the person, the wrist/arm band 290 presses the artery to block the blood from flowing through the wrist or the arm. Therefore, the heartbeat detector 260 arranged in the wrist/arm band 290 does not output the pulsation signal.

When the pressure of the main air tank 210 is released and lower than the blood pressure of the user, the blood in the artery is ejected from the pressed position of the wrist/arm band 290 to form the eddy current and thus generate Korothov sound. The heartbeat detector 260 outputs several pulsation signals due to the artery beats of the user.

At the point A shown in FIG. 3, when the pulsation signal is Korothov sound, the controller 270 activates the valve 225 to release air in the auxiliary air tank 220 into the main air tank 210. The controller 270 sets the pressure value outputted by the pressure detector 240 as an approximate systolic pressure (Pₐ₋ₕₚ) and opens the valve 225 to release air in the auxiliary air tank 220 into the main air tank 210. The pressure increases in the main air tank 210 with a first slow slope S_{slow1}, wherein the absolute value of the first slow slope S_{slow1} is lower than that of the first fast slope S_{fast1} so as to measure the systolic pressure of the user accurately.

At the point B shown in FIG. 3, when the Korothov sound disapears, the controller 270 sets the pressure value outputted by the pressure detector 240 as an accurate systolic pressure (Pₕₚ) and closes the valve 225. When receiving the accurate systolic pressure (Pₕₚ), the controller 270 activates the pressure release device 240 to decrease the pressure of the main air tank 210 with a second fast slope S _{fast2}. When the pressure in the main air tank 210 is lower than the approximate systolic pressure (Pₐ₋ₕₚ), the heartbeat detector 260 detects Korothov sound again, and the pressure in the main air tank 210 decreases gradually.

At the point C shown in FIG. 3, when the Korothov sound disapears again, the controller 270 sets the pressure value outputted by the pressure detector 240 as an approximate diastolic blood pressure (Pₐ₋ₗₚ) and opens the valve 225 to release air in the auxiliary air tank 220 into the main air tank 210 resulting in increasing the pressure in the main air tank 210 with a second slow slope S_{slow2}.

At the point D shown in FIG. 3, when the Korothov sound appears, the controller 270 sets the pressure value outputted by the pressure detector 240 as an accurate diastolic blood pressure (Pₗₚ) and closes the valve 225. The controller 270 releases the pressure in the main air tank 210 and the auxiliary air tank 220.

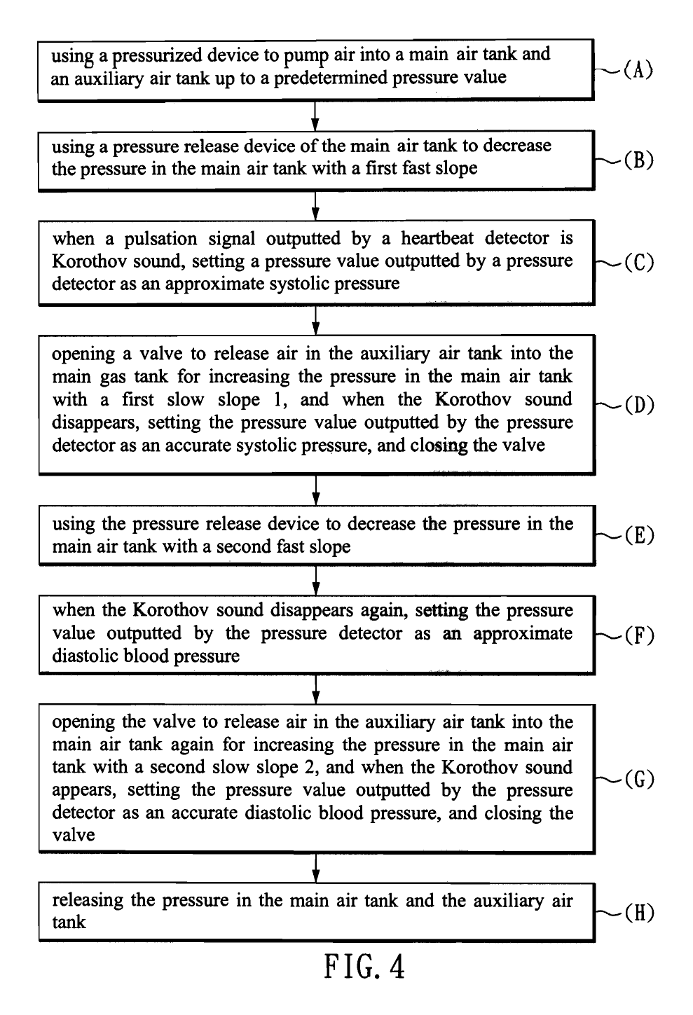

With reference to FIG. 4, there is shown a flow chart of high-accuracy blood pressure measuring method for measuring the blood pressure of a user. Please also refer to the pressure diagram of air being released from the main air tank 210 in FIG. 3.

First, in the step (A), a pressurized device 230 is used to pump air into a main air tank 210 and a auxiliary air tank 220 up to a predetermined pressure value P_{H} (i.e. 220 mmHg).

In the step (B), a pressure release device 240 of the main air tank 210 is used to decrease the pressure in the main air tank 210 with a first fast slope (S_{fast1}).

In the step (C), when a pulsation signal outputted by a heartbeat detector 260 is Korothov sound, a pressure value outputted by a pressure detector 250 is set as an approximate systolic pressure (Pₐ₋ₕₚ).

In the step (D), a valve 225 is opened to release air in the auxiliary air tank 220 into the main air tank 210 for increasing the pressure in the main air tank 210 with a first slow slope (S_{slow1}), and when the Korothov sound disappears, the pressure value outputted by the pressure detector 250 is set as an accurate systolic pressure (Pₕₚ), and the valve 225 is closed.

In the step (E), the pressure release device 240 is used to decrease the pressure in the main air tank 210 with a second fast slope (S_{fast2}).

In the step (F), when the Korothov sound disappears again, the pressure value outputted by the pressure detector 250 is set as an approximate diastolic blood pressure (Pₐ₋ₗₚ).

In the step (G), the valve 225 is opened to release air in the auxiliary air tank 220 into the main air tank 210 again for increasing the pressure in the main air tank 210 with a second slow slope (S_{slow2}), and when the Korothov sound appears, the pressure value outputted by the pressure detector 250 is set as an accurate diastolic blood pressure (Pₗₚ), and the valve 225 is closed.

In the step (H), the pressure in the main air tank 210 and the auxiliary air tank 220 is released.

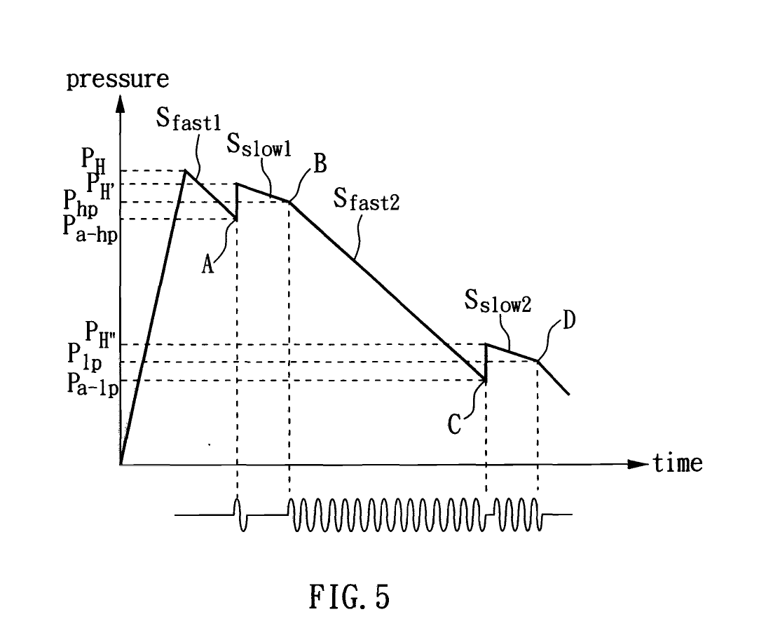

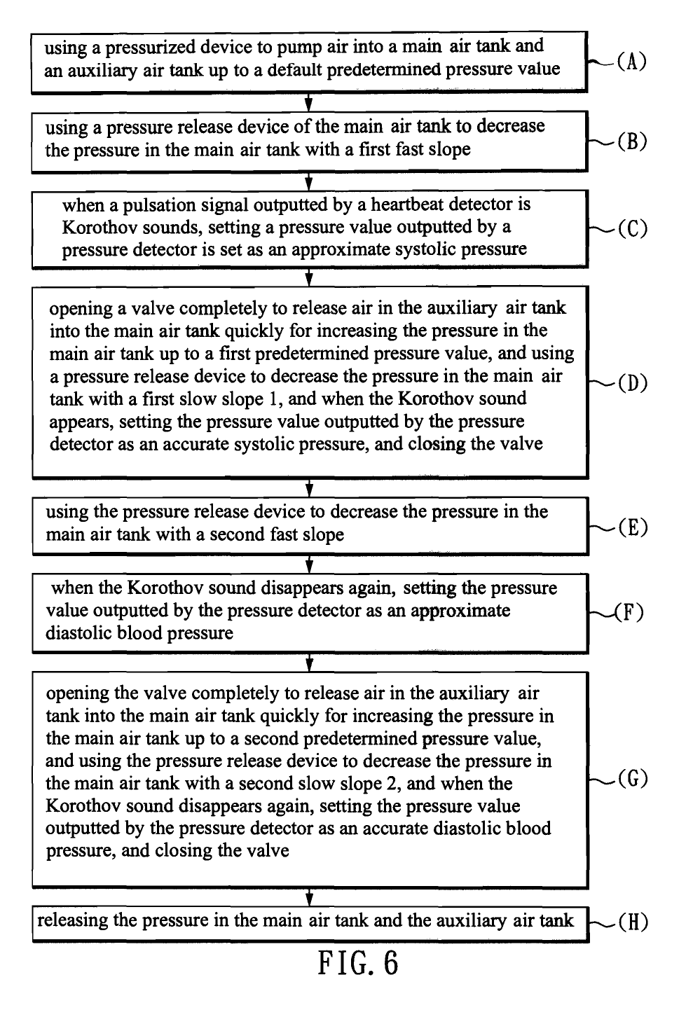

FIG. 5 shows a pressure diagram of air being released from the main air tank 210 in accordance with another embodiment of the present invention. FIG. 6 shows a flow chart of high-accuracy blood pressure measuring method in accordance with another embodiment of the present invention. Please refer to FIG. 5 and FIG. 6 at the same time. The step (A) to step (C) in the FIG. 6 is the same with the step (A) to step (C) in the FIG. 4.

When the controller 270 receives the approximate systolic pressure (Pₐ₋ₕₚ), in the step (D), the controller 270 opens a valve 225 completely to release air in the auxiliary air tank 220 into the main air tank 210 rapidly for increasing the pressure in the main air tank 210 up to a first predetermined pressure value P_{H′}, wherein the first predetermined pressure value P_{H′} is higher than the approximate systolic pressure (Pₐ₋ₕₚ). Due to that the pressurized device 230 is not activated, the air in the auxiliary air tank 220 is released into the main air tank 210 rapidly so that the first predetermined pressure value P_{H′} is lower than the predetermined pressure value P_{H}.

The controller 270 uses the pressure release device 240 in the main air tank 210 to decrease the pressure in the main air tank 210 with a first slow slope (S_{slow1}). When the Korothov sound appears, the controller 270 sets the pressure value outputted by the pressure detector 250 as an accurate systolic pressure (Pₕₚ) and closes the valve 225, wherein the absolute value of the first slow slope S_{slow1} is lower than that of the first fast slope S_{fast1} so as to measure the systolic pressure of the user accurately.

The step (E) in FIG. 6 is the same with the step (E) in FIG. 4. The pressure release device 240 is used to decrease the pressure in the main air tank 210 with a second fast slope (S_{fast2}).

The step (F) in FIG. 6 is the same with the step (F) in FIG. 4. When the Korothov sound disappears, the controller 270 sets the pressure value outputted by the pressure detector 250 as an approximate diastolic blood pressure (Pₐ₋ₗₚ).

In the step (G), the controller 270 opens the valve 225 completely to release air in the auxiliary air tank 220 into the main air tank 210 quickly for increasing the pressure in the main air tank 210 up to a second predetermined pressure value P _{H″}. The controller 270 activates the pressure release device 240 to decrease the pressure in the main air tank 210 with a second slow slope (S_{slow2}). When the Korothov sound disappears again, the controller 270 sets the pressure value outputted by the pressure detector 250 as an accurate systolic pressure (Pₕₚ), wherein second predetermined pressure value P_{H″} is higher than the approximate diastolic blood pressure (Pₐ₋ₗₚ).

In the step (H), the controller 270 releases the pressure in the main air tank 210 and the auxiliary air tank 220.

According to the above description, using the auxiliary air tank 220 without activating the pressurized device 230 again in the present invention can avoid additional power loss resulting from frequent starts of the pressurized device 230 without generating noise. Therefore, the present invention is especially suitable for battery-powered handheld hemadynamometer. When measuring blood pressure, the main air rank 210 decreases the pressure in the main air tank 210 with higher speed (S_{fast}) in order to save measuring time. Due to using the auxiliary air tank 220, the pressure in the main air tank 210 can increase the pressure in the main air tank 210 close to the diastolic blood pressure and systolic pressure of the user with slower speed (S_{slow}), or rapidly increase the pressure in the main air tank 210 and slowly decrease the pressure in the main air tank 210 later to measure the blood pressure of the user accurately. The present invention is characterized in using high slope to get measuring speed and using slow slope to get accuracy. The auxiliary air tank 220 can also be arranged in the wrist/arm band 290. Due to that the auxiliary air tank 220 is made from a rigid container, the pressure of the auxiliary air tank 220 does not affect the pressure of the wrist/arm band 290 directly. The auxiliary air tank 220 may also not directly connected to the pressurized device 230, and the valve 225 is open in the step (A) of FIG. 4 and FIG. 6 until the step (A) is over, which can achieve the same function.

Although the present invention has been explained in relation to its preferred embodiment, it is to be understood that many other possible modifications and variations can be made without departing from the scope of the invention as hereinafter claimed.

What is claimed is:

1. A high-accuracy hemadynamometer for measuring the blood pressure of a user, comprising:
a main air tank for imposing pressure upon a wrist or an arm of the user;
an auxiliary air tank connected to the main air tank, wherein a valve is set between the auxiliary air tank and the main air tank to release air in the auxiliary air tank into the main air tank;
a pressurized device connected to the main air tank and the auxiliary air tank for pumping air into the main air tank and the auxiliary air tank to impose pressure upon the wrist or the arm of the user;
a pressure release device arranged on the main air tank;
a pressure detector connected to the main air tank for detecting the pressure of the main air tank and outputting a pressure value;
a heartbeat detector for detecting artery beats of the user and generating a pulsation signal corresponding to the heartbeats; and
a controller connected to the pressure detector, the heartbeat detector, the pressure release device and the valve for controlling the pressure release device to release the pressure in the main air tank, and activating the valve based on the pulsation signal to release air in the auxiliary air tank into the main air tank.

2. The high-accuracy hemadynamometer of claim 1, wherein when the pulsation signal is Korothov sound, the controller activates the valve to release air in the auxiliary air tank into the main air tank.

3. The high-accuracy hemadynamometer of claim 2, wherein the pressurized device pumps air into the main air tank and the auxiliary air tank up to a predetermined pressure value, and the controller activates the pressure release device to decrease the pressure in the main air tank with a first fast slope.

4. The high-accuracy hemadynamometer of claim 3, wherein when the pulsation signal is Korothov sound, the controller sets the pressure value outputted by the pressure detector as an approximate systolic pressure, and activates the valve to release air in the auxiliary air tank into the main air tank so as to increase the pressure in the main air tank with a first slow slope; when the Korothov sound disapears, the controller sets the pressure value outputted by the pressure detector as an accurate systolic pressure and closes the valve.

5. The high-accuracy hemadynamometer of claim 4, wherein when receiving the accurate systolic pressure, the controller activates the pressure release device to decrease the pressure of the main air tank with a second fast slope; when the Korothov sound disappears again, the controller sets the pressure value outputted by the pressure detector as an approximate diastolic blood pressure and opens the valve to release air in the auxiliary air tank into the main air tank so as to increase the pressure in the main air tank with a second slow slope; when the Korothov sound appears, the controller sets the pressure value outputted by the pressure detector as an accurate diastolic blood pressure and closes the valve.

6. The high-accuracy hemadynamometer of claim 2, further comprising:
a display unit connected to the controller for displaying the pressure value outputted by the pressure detector.

7. The high-accuracy hemadynamometer of claim 6, wherein the pressure release device is a motor or an air pump.

8. The high-accuracy hemadynamometer of claim 7, wherein the pressure release device is an air releasing valve.

9. A high-accuracy blood pressure measuring method for measuring the blood pressure of a user, comprising the steps of:
(A) using a pressurized device to pump air into a main air tank and a auxiliary air tank up to a predetermined pressure value;
(B) using a pressure release device of the main air tank to decrease the pressure in the main air tank with a first fast slope;
(C) when a pulsation signal outputted by a heartbeat detector is Korothov sound, setting a pressure value outputted by a pressure detector as an approximate systolic pressure; and
(D) opening a valve to release air in the auxiliary air tank into the main air tank for increasing the pressure in the main air tank with a first slow slope, and when the Korothov sound disappears, setting the pressure value outputted by the pressure detector as an accurate systolic pressure, and closing the valve.

10. The high-accuracy blood pressure measuring method of claim 9, further comprising the steps of:
(E) using the pressure release device to decrease the pressure in the main air tank with a second fast slope;
(F) when the Korothov sound disappears again, setting the pressure value outputted by the pressure detector as an approximate diastolic blood pressure;
(G) opening the valve to release air in the auxiliary air tank into the main air tank again for increasing the pressure in the main air tank with a second slow slope, and when the Korothov sound appears, setting the pressure value outputted by the pressure detector as an accurate diastolic blood pressure, and closing the valve; and
(H) releasing the pressure in the main air tank and the auxiliary air tank.

11. A high-accuracy blood pressure measuring method for measuring the blood pressure of a user, comprising the steps of:
(A) using a pressurized device to pump air into a main air tank and a auxiliary air tank up to a predetermined pressure value;
(B) using a pressure release device of the main air tank to decrease the pressure in the main air tank with a first fast slope;
(C) when a pulsation signal outputted by a heartbeat detector is Korothov sound, setting a pressure value outputted by a pressure detector as an approximate systolic pressure;
(D) opening a valve completely to release air in the auxiliary air tank into the main air tank quickly for increasing the pressure in the main air tank up to a first predetermined pressure value, and using a pressure release device to decrease the pressure in the main air tank with a first slow slope, and when the Korothov sound appears, setting the pressure value outputted by the pressure detector as an accurate systolic pressure, and closing the valve;
(E) using the pressure release device to decrease the pressure in the main air tank with a second fast slope;
(F) when the Korothov sound disappears again, setting the pressure value outputted by the pressure detector as an approximate diastolic blood pressure;
(G) opening the valve completely to release air in the auxiliary air tank into the main air tank quickly for increasing the pressure in the main air tank up to a second predetermined pressure value, and using the pressure release device to decrease the pressure in the main air tank with a second slow slope, and when the Korothov sound disappears again, setting the pressure value outputted by the pressure detector as an accurate diastolic blood pressure, and closing the valve; and
(H) releasing the pressure in the main air tank and the auxiliary air tank.
